(19) United States
(12) Patent Application Publication
(10) Pub. No.: US 2025/0263354 A1
(43) Pub. Date: Aug. 21, 2025

(54) BUTADIENE PRODUCTION FROM USED TIRES

(71) Applicant: Bridgestone Americas Tire Operations, LLC, Nashville, TN (US)

(72) Inventors: Terrence E. HOGAN, Akron, OH (US); William S. NIAURA, Akron, OH (US); Mark W. SMALE, Akron, OH (US)

(21) Appl. No.: 18/856,238

(22) PCT Filed: Apr. 12, 2023
(86) PCT No.: PCT/US2023/065644

§ 371 Date: Oct. 11, 2024

Related U.S. Application Data
(60) Provisional application No. 63/330,240, filed on Apr. 12, 2022.

(51) Int. Cl. C07C 5/48; B60C 1/00; C07C 1/20; C07C 29/151; C08F 236/06; C08J 11/12

(52) U.S. Cl. CPC C07C 5/48; B60C 1/00; C07C 1/20; C07C 29/1518; C08F 236/06; C08J 11/12; C08J 2317/00

(57) ABSTRACT

A process comprising (a) providing a feedstock that includes carbonaceous materials; (b) gasifying the feedstock to produce a gaseous stream including carbon monoxide, hydrogen, and carbon dioxide; (c) converting at least a portion of the carbon monoxide, hydrogen, and carbon dioxide to methanol; (d) converting at least a portion of the methanol to butene; and (e) converting at least a portion of the butene to butadiene monomer.

### FIELD OF THE INVENTION

Embodiments of the present invention are directed toward a process for converting used tires to butadiene monomer.

### BACKGROUND OF THE INVENTION

Butadiene monomer is polymerized to polybutadiene and butadiene copolymers such as poly(styrene-co-butadiene), poly(isoprene-co-butadiene), and poly(styrene-co-isoprene-co-butadiene). While these polymers have many uses, they are significantly used in the manufacture of tires. Used tires, on the other hand, are not easily recycled and have been landfilled or incinerated for fuel value. Methods have been proposed to thermally decompose waste, including tires into syngas, and then convert the syngas to useful materials. These methods lack industrial applicability, and therefore improvements to this general pathway are desired.

### SUMMARY OF THE INVENTION

One or more embodiments of the present invention provide a process comprising (a) providing a feedstock that includes carbonaceous materials; (b) gasifying the feedstock to produce a gaseous stream including carbon monoxide, hydrogen, and carbon dioxide; (c) converting at least a portion of the carbon monoxide, hydrogen, and carbon dioxide to methanol; (d) converting at least a portion of the methanol to butene; and (e) converting at least a portion of the butene to butadiene monomer.

Other embodiments of the present invention provide a method for producing an elastomeric polymer, the method comprising (a) providing 1,3-butadiene monomer synthesized from constituents of a gaseous stream that is obtained from the gasification of a feedstock including carbonaceous materials; and (b) polymerizing the 1,3-butadiene monomer to form a polymer.

Yet other embodiments of the present invention provide a tire component comprising a cured rubber matrix with filler dispersed therein, where the cured rubber matrix includes greater than 40 wt %, or 50 wt %, or 60 wt %, or 70 wt %, or 80 wt %, or 90 wt %, or 99 wt % of a crosslinked circular synthetic rubber that is obtained by polymerizing monomer synthesized from constituents of a gaseous stream that is obtained from the gasification of a feedstock including carbonaceous materials.

### DETAILED DESCRIPTION OF ILLUSTRATIVE EMBODIMENTS

Embodiments of the invention are based, at least in part, on the discovery of a process for consuming used tires in the production of butadiene monomer (e.g. 1,3-butadiene). The butadiene monomer can advantageously be polymerized to polybutadiene or butadiene copolymers, and tire components can be produced from the polybutadiene or butadiene copolymers. Embodiments of the invention therefore provide a methodology for converting used tires back to useful tires or tire components. According to one or more embodiments, used tires are thermally decomposed to form a gaseous stream. One or more components of this gaseous stream are then converted to butadiene monomer. In one or more embodiments, one or more components of the gaseous stream are converted to methanol, and the methanol is then converted to butadiene. In one or more embodiments, the conversion of methanol to butadiene can include intermediary conversion of the methanol to butene, and then the butene is converted to butadiene.

### Process Overview

As suggested above, the process of the present invention provides a feedstock, which may include tire feedstock, and the feedstock is converted, via thermal decomposition, to a gaseous stream that includes hydrogen, carbon monoxide, and optionally carbon dioxide. This gaseous stream, which may be referred to as synthesis gas or syngas, is then converted to methanol, which is contained within a methanol-containing stream. The methanol within the methanol-containing stream is then converted to butene within a butene-containing stream, and then the butene within the butene-containing stream is converted to butadiene contained within a butadiene-containing stream.

### Characteristics of Feedstock

In one or more embodiments, the feedstock that is thermally decomposed to form the gaseous stream may include tire feedstock from used tires, which may also be referred to as used tire feedstock. As the skilled person appreciates, tire feedstock may include vulcanized polymer, carbon black filler, silica, resins, oils, fibrous yarn, and metal. The vulcanized polymer may include the sulfur-crosslinked residue of natural rubber and/or one or more synthetic elastomers including diene polymers and copolymers. In one or more embodiments, the used tire feedstock may include shredded or otherwise ground tires with one or more constituents of the used tire removed. For example, the tire feedstock may be treated to remove metal by methods known in the art (e.g. magnetic separation). Alternatively, or in combination therewith, the used tire feedstock may be optionally treated to remove fibrous reinforcement such as fiber yarn or cord, which the skilled person understands is often found in conjunction with the vulcanized rubber within many tire components. Alternatively, or in combination with the foregoing, the used tire feedstock may be optionally treated to remove inorganic materials such as silica filler, which the skilled person appreciates is often found used tire components. In any event, the tire feedstock can be processed into tire shreds, tire chips, or ground or crumb rubber and fed to the thermal decomposition unit.

In one or more embodiments, the tire feedstock is characterized by relatively low amounts of metal, which may result from pre-treatment of the tire feedstock to remove metal. In one or more embodiments, the tire feedstock includes less than 25 wt %, in other embodiments less than 15 wt %, and in other embodiments less than 1 wt % metal based on the entire weight of the feedstock fed to thermal decomposition in accordance with the present invention.

In one or more embodiments, the tire feedstock is characterized by relatively low amounts of fibrous yarn or cord, which may result from pre-treatment of the tire feedstock to remove fibrous yarn or cord. In one or more embodiments, the tire feedstock includes less than 5 wt %, in other embodiments less than 4 wt %, in other embodiments less than 3 wt %, in other embodiments less than 2 wt %, and in other embodiments less than 1 wt % fibrous yarn or cord based on the entire weight of the feedstock fed to thermal decomposition in accordance with the present invention.

In one or more embodiments, the tire feedstock is characterized by relatively low amounts of inorganic filler (e.g. silica), which may result from pre-treatment of the tire feedstock to remove inorganic filler. In one or more embodiments, the tire feedstock includes less than 30 wt %, in other embodiments less than 20 wt %, in other embodiments less than 10 wt %, and in other embodiments less than 5 wt % inorganic filler based on the entire weight of the feedstock fed to thermal decomposition in accordance with the present invention.

In one or more embodiments, the used tire feedstock includes tire remains from passenger tires. In other embodiments, the used tire feedstock includes tire remains from non-passenger tires such as, but not limited to, truck and bus tires, off-road vehicle tires, agricultural tires, and race tires.

In one or more embodiments, the used tires may be characterized by a compacted density of greater than 640 kg/m³, in other embodiments greater than 720 kg/m³, and in other embodiments greater than 770 kg/m³, where density is determined by ASTM D 698-07.

In one or more embodiments, the feedstock provided to the thermal decomposition unit includes tire feedstock and optionally complementary feedstock. In one or more embodiments, the complementary feedstock, which may also be referred to as co-feed, includes carbonaceous materials other than the tire feed stock. Carbonaceous material refers to any carbon material whether in solid, liquid, gas, or plasma state. Non-limiting examples of carbonaceous materials include carbonaceous liquid product, industrial liquid recycle, municipal solid waste (MSW or msw), urban waste, agricultural material, forestry material, wood waste, construction material, vegetative material, industrial waste, fermentation waste, petrochemical coproducts, alcohol production coproducts, coal, plastics, waste plastic, coke oven tar, lignin, black liquor, polymers, waste polymers, polyethylene terephthalate (PETA), polystyrene (PS), sewage sludge, animal waste, crop residues, energy crops, forest processing residues, wood processing residues, livestock wastes, poultry wastes, food processing residues, ethanol coproducts, spent grain, spent microorganisms, municipal waste, construction waste, demolition waste, biomedical waste, hazardous waste, or their combinations. In one or more embodiments, the carbonaceous material includes biomass. In one or more embodiments, the biomass is bagasse including, but not limited to, the bagasse of sugar cane, sorghum, and guayule plant.

In a further embodiment, the guayule bagasse is produced as the result of a process to extract rubber and resin from the guayule plant, such as described in U.S. Publication No. 2022/0356273, which is incorporated herein by reference. Methods for the desolventization of guayule bagasse are described in U.S. Pat. No. 10,132,563, which is also incorporated herein by reference. In one or more embodiments, the guayule bagasse contains no more than 1 wt % organic solvent (based upon the total weight of the dried bagasse). In certain embodiments, the dried bagasse contains no more than 0.5 wt % organic solvent (based upon the total weight of the dried bagasse). In one or more embodiments, the dried bagasse may contain a quantity of water and higher boiling point terpenes. In certain embodiments, the total quantity of water and higher boiling point terpenes in the dried bagasse may be higher than the content of organic solvents. In certain embodiments, resin content (including the higher boiling point terpenes) in the dried bagasse is generally acceptable and, in some instances, actually preferred.

In one or more embodiments, the co-feed (e.g. biomass or municipal waste) may be characterized by a compacted density of less than 600 kg/m³, in other embodiments less than 580 kg/m³, and in other embodiments less than 560 kg/m³, where density is determined by ASTM D 698-07.

The feedstock may be characterized by the amount of co-feed (e.g. biomass or municipal waste). In one or more embodiments, the feedstock includes from about 0 to about 95, in other embodiments from about 1 to about 75, and in other embodiments from about 2 to about 55 wt % co-feed with the balance including used tire. In one or more embodiments, the feedstock includes less than 95, in other embodiments less than 80, and in other embodiments less than 70 wt % co-feed. In these or other embodiments, the feedstock includes greater than 10, in other embodiments greater than 20, in other embodiments greater than 30, in other embodiments greater than 40, in other embodiments greater than 50, and in other embodiments greater than 70 wt % used tires, with the balance including complementary feedstock.

In alternative embodiments, the feedstock is substantially, and in certain embodiments exclusively, comprised of those carbonaceous materials identified above other than tire feedstock; i.e. the feedstock is substantially or exclusively comprised of the co-feed materials identified above. For example, in one or more embodiments, the feedstock includes greater than 80 wt %, in other embodiments greater than 90 wt %, and in other embodiments greater than 99 wt % municipal solid waste. Or, in other exemplary embodiments, the feedstock includes greater than 80 wt %, in other embodiments greater than 90 wt %, and in other embodiments greater than 99 wt % biomass.

### Thermal Decomposition of Feedstock

As indicated above, the feedstock (which may include tire feedstock and optionally co-feed) is thermally decomposed into gaseous streams including hydrogen, carbon monoxide, and optionally carbon dioxide by employing techniques that are generally known in the art. As the skilled person understands, these processes may include gasification processes, and it is also known that these processes can be tailored to control the chemical nature of the resulting gaseous stream. For example, the degree of combustion can be controlled by controlling the amount of oxygen present during thermal decomposition. In one or more embodiments, the step of thermal decomposition takes place in a substantially inert environment.

Processes that may be used for the thermal decomposition step may include pyrolysis or gasification reactions as disclosed in U.S. Publication Nos. 2021/0207037; 2019/0295734; 2019/0249089; 2018/0273415; 2017/0009162; 2017/0002271; 2016/0107913; 2016/0068773; 2016/0024404; 2014/0182205; 2014/0157667; and 2014/0100294, which are incorporated herein by reference.

In one or more embodiments, where the feedstock includes both tire feedstock and co-feed, the tire feedstock and the co-feed can be introduced to the same thermal decomposition unit simultaneously. For example, the tire feedstock and the co-feed can be pre-mixed at a desired ratio to form the feedstock that is fed to the thermal decomposition unit. Alternatively, separate streams of tire feedstock and co-feed can be separately and individually fed to the thermal decomposition unit at a desired rate. In yet other embodiment, the two feedstocks (i.e. the tire feedstock and the co-feed) can be sequentially treated within the same thermal decomposition unit. In still other embodiments, the two feedstocks (i.e. the tire feedstock and the co-feed) can be treated within separate thermal decomposition units operating in parallel, and then the gaseous streams produced by the respective units can be combined to attain the desired ratio of gaseous constituents.

### Characteristics of Gaseous Product Stream

As indicated above, the gaseous product stream produced by thermal decomposition of the feedstock includes carbon monoxide, hydrogen and optionally carbon dioxide. In one or more embodiments, the gaseous product stream includes from about 5 to about 50, or in other embodiments from about 7 to about 25, or in other embodiments from about 8 to about 15 volume percent carbon dioxide. In these or other embodiments, the gaseous product stream includes from about 10 to about 85, or in other embodiments from about 20 to about 65, or in other embodiments from about 25 to about 45 volume percent hydrogen. In these or other embodiments, the gaseous product stream includes from about 20 to about 85, or in other embodiments from about 30 to about 75, or in other embodiments from about 40 to about 60 volume percent carbon monoxide. In one or more embodiments, the gaseous product stream produced by thermal decomposition includes from about 40 to about 80 wt %, in other embodiments from about 45 to about 75 wt %, and in other embodiments from about 50 to about 70 wt % carbon (i.e. carbon within carbon-based compounds) based on the total weight of the gaseous product stream.

### Syngas to Methanol

As indicated above, constituents of the gaseous stream are converted to methanol that is contained within a methanol-containing stream; i.e. a product stream that includes methanol. In one or more embodiments, the gaseous stream is supplemented with hydrogen prior to converting the gaseous stream to a methanol-containing stream. Also, the gaseous stream can be treated to remove undesirable constituents prior to converting the gaseous stream to a methanol-containing stream. For example, the skilled person understands that certain additives can be introduced to the gaseous stream to thereby remove (e.g. scavenge) sulfur and other impurities.

In one or more embodiments, the gaseous stream is converted to a methanol-containing stream by catalytic conversion of hydrogen and carbon monoxide to methanol. Known methods for this gas-phase process can be used. In one or more embodiments, this gas-phase process can be carried out within a fixed-bed reactor at a relatively high pressure. In one or more embodiments, the gas-phase conversion of hydrogen and carbon monoxide may be carried out at pressures from about 600 psig to about 1,700 psig. In one or more embodiments, the gas-phase conversion of hydrogen and carbon monoxide may be carried out at temperatures from about 400° F. to about 600° F.

In one or more embodiments, useful catalysts for converting hydrogen and carbon monoxide to methanol may include one or more of copper, zinc oxide, alumina, magnesia, and mixtures thereof. Another exemplary catalyst is one including carbon, nitrogen, and platinum.

Processes for converting hydrogen and carbon monoxide to methanol are commercially available. For example, processes and systems that include liquid phase methanol (LPMEOH™) are marketed by Air Products and Chemicals, Inc.

In one or more embodiments, a crude methanol-containing stream obtained directly from the reactor used to produce the methanol is delivered to downstream steps. In other embodiments, the methanol-containing stream is purified to produce a methanol-containing stream that is higher in methanol content. In one or more embodiments, the methanol-containing stream exiting the step wherein syngas is converted to methanol and delivered to the downstream steps where methanol is converted (optionally after purification) includes greater than 80 wt %, in other embodiments greater than 90 wt %, and in other embodiments greater than 95 wt % methanol based upon the entire weight of the methanol-containing stream.

### Methanol to Butene

As indicated above, methanol within the methanol-containing stream is converted to an alkene (i.e. an olefin), particularly butene, which product stream may be referred to as a butene-containing stream. In one or more embodiments, this is accomplished by using the methanol-to-olefins (MTO) process. As the skilled person understands, MTO processes convert methanol to olefins, which can include ethylene, propylene, and butene. Conventional MTO processes can produce a wide range of olefins, and the skilled person understands that MTO processes can be tailored to preferentially produce larger percentages of a targeted product, such as butene. For example, it is well-known that selectivity toward certain species, such as butene, can be targeted by adjusting factors such temperature and pressure, as well as other factors such catalyst selection.

In one or more embodiments, the MTO process employed in the present invention includes an acidic zeolite catalyst. In an exemplary process, the methanol-containing stream is fed to a reactor containing the desired catalyst (e.g. acidic zeolite catalyst). The reactor may be coupled to a regenerator for regenerating the catalyst. After converting the methanol to olefins, the reaction product stream (i.e. the butene-containing stream) may be subjected to a water removal step. This may be accomplished by employing distillation techniques. The desired olefins are typically separated as an overhead stream, which may then be subjected to one or more olefin separation processes, which may include the use of distillation techniques. The one or more olefin separation steps may include separate steps for the removal of C1 carbons (e.g. methylene, methane), ethylene, propylene, and a C4+ product (e.g. butene). The butene may be separated from other C4+ carbons.

As alluded to above, the temperature of the MTO process can be manipulated to target preferential production of butene. In one or more embodiments, the temperature of the MTO process may be at least 300° C., in other embodiments at least 325° C., in other embodiments at least 350° C., and in other embodiments at least 375° C. In one or more embodiments, the temperature of the MTO process may be from about 300° C. to about 400° C., in other embodiments, from about 300° C. to about 375° C., in other embodiments, from about 325° C. to about 375° C., and in other embodiments, from about 350° C. to about 375° C.

Commercial processes are known for converting methanol to butene. For example, commercial processes are available from Honeywell under the tradename UOP Advanced MTO (Methanol to Olefins) Technology.

In one or more embodiments, a crude butene-containing stream obtained directly from the reactor used to produce the butene is delivered to downstream steps. In other embodiments, the butene-containing stream is purified to produce a butene-containing stream that is higher in butene content; for example, the olefin product stream is subjected to distillation as described above. In one or more embodiments, the butene-containing stream exiting the step wherein methanol is converted to butene and delivered to the downstream steps (e.g. optionally purified) where butene is converted includes greater than 80 wt %, in other embodiments greater than 90 wt %, and in other embodiments greater than 95 wt % butene based upon the entire weight of the methanol-containing stream.

### Butene to Butadiene

As indicated above, butene within the butene-containing stream is converted to butadiene. Processes for converting butene to butadiene, particularly 1,3-butadiene, are known. For example, an exemplary commercial process is available from Honeywell under the tradename UOP/TPC Group OXO-D™ Technology. This technology generally includes conversion of butene into butadiene through oxidative dehydrogenation. This may include the use of OXO-D™ catalyst available from Honeywell. Processes for the conversion of butene to butadiene are also known in the literature; for example, these processes are disclosed in U.S. Pat. No. 10,052,608, which is incorporated herein by reference.

In one or more embodiments, the product stream obtained from the step of converting butene to butadiene is a crude stream obtained directly from the reactor used to produce the butadiene. In other embodiments, the butadiene-containing stream is purified to produce a butadiene-containing stream that is higher in butadiene content; for example, the butadiene-containing stream can be subjected to distillation as described above. In one or more embodiments, the butadiene-containing product stream obtained from this step (e.g. optionally purified) includes greater than 80 wt %, in other embodiments greater than 90 wt %, and in other embodiments greater than 95 wt % butene based upon the entire weight of the methanol-containing stream.

### INDUSTRIAL APPLICABILITY

In one or more embodiments, the butadiene monomer (e.g. 1,3-butadiene) produced by the methods of this invention can be used in the production of polybutadiene or butadiene copolymers (which may also be referred to as polybutadiene copolymers). For purposes of this specification, these polymers may be referred to as circular synthetic rubber, or circular synthetic polybutadiene to butadiene copolymers. In one or more embodiments, this circular synthetic rubber can be used in the manufacture of tire components. As a result, practice of the present invention provides a method by which waste material, in particular that waste material from used tires, is converted back to useful tires. In other words, a tire recycling or tire circularity method is provided.

The synthesis of polybutadiene or polybutadiene copolymers from butadiene monomer is well known and can be accomplished by using several synthetic routes (i.e. polymerization mechanisms and techniques). For example, the monomer can be polymerized by free-radical emulsion polymerization, anionic polymerization, or coordination catalysis using, for example, nickel or neodymium-based catalyst systems.

As the skilled person appreciates, comonomers that can be copolymerized with butadiene to form polybutadiene copolymers include, but are not limited to, vinyl aromatic monomer such as styrene, as well as other diene monomer such as isoprene. In one or more embodiments, the comonomer is a sustainable comonomer. For example, styrene can be obtained from bio-synthesized feedstock, such as bioethanol, that is subsequently converted to styrene; see, e.g. U.S. Pat. No. 9,663,445. Alternatively, styrene can be synthesized from bio-based materials such as cinnamic acid or hydrocinnamic acid; see, U.S. Pat. No. 9,868,853. Yet other examples include styrene obtained from the depolymerization of polystyrene from post-consumer waste; see U.S. Publication No. 2022/0411351. The skilled person also understands that styrene can be obtained from those processes that are mass balanced to qualify as bio-based, bio-circular, or circular as designated by the International Sustainability and Carbon Certification ISCC.

It should be appreciated that the polybutadiene polymers prepared by polymerizing the butadiene of the present invention have a relatively high percentage of mer units deriving from the butadiene produced by the invention, and therefore the polybutadiene polymers of this invention have a relatively high sustainable content. In one or more embodiments, the polybutadiene polymers synthesized by polymerizing the butadiene of the present invention include greater than 50 mol %, in other embodiments greater than 60 mol %, in other embodiments greater than 70 mol %, in other embodiments greater than 80 mol %, in other embodiments greater than 90 mol %, in other embodiments greater than 95 mol %, and in other embodiments greater than 99 mol % sustainable mer units, which obtained from polymerization of butadiene obtained by the present invention (i.e. mer units obtained from monomer that is synthesized from a gaseous stream obtained by gasification of carbonaceous materials). Likewise, where the polymer synthesized is a polybutadiene copolymer by copolymerizing the butadiene monomer obtained by practice of this invention together with sustainable comonomer, the resulting polybutadiene copolymer has a relatively high sustainable content. In one or more embodiments, the polybutadiene copolymers synthesized by polymerizing the butadiene of the present invention together with sustainable comonomer include greater than 50 mol %, in other embodiments greater than 60 mol %, in other embodiments greater than 70 mol %, in other embodiments greater than 80 mol %, in other embodiments greater than 90 mol %, in other embodiments greater than 95 mol %, and in other embodiments greater than 99 mol % sustainable mer units (i.e. mer units that are obtained from monomer synthesized from a gaseous stream obtained by gasification of carbonaceous materials and other sustainable comonomer).

The polymers synthesized from butadiene monomer produced by embodiments of the invention may be referred to as vulcanizable polymers, or as elastomeric polymers, and generally include polydienes and polydiene copolymers. Specific of polymers that can be produced and used in the manufacture of tires include, but are not limited to, polybutadiene, poly(styrene-co-butadiene), poly(styrene-co-isoprene-co-butadiene), poly(isoprene-co-butadiene), and functionalized derivatives thereof.

The polybutadiene and polybutadiene copolymers produced by the present invention exhibit excellent viscoelastic properties and are particularly useful in the manufacture of various tire components including, but not limited to, tire treads, sidewalls, subtreads, and bead fillers. These polymers can be used as all or part of the elastomeric component of a tire stock. When the polymers produced by the present invention are used in conjunction with other vulcanizable polymers to form the elastomeric component of a tire stock, these other vulcanizable polymers may include natural rubber, synthetic rubbers, and mixtures thereof. Examples of synthetic rubber include polyisoprene, poly(styrene-co-butadiene), and other polybutadienes with low and/or cis-1,4-linkage content, poly(styrene-co-butadiene-co-isoprene), and mixtures thereof. The polymers of this invention can also be used in the manufacture of hoses, belts, shoe soles, window seals, other seals, vibration damping rubber, and other industrial products.

Practice of the present invention not only offers a method for recycling tires by employing used tires as a feedstock to produce polymer that can be formulated back into tires, but the practice of the present invention also advantageously provides a method whereby a tire is produced that has a relatively high content of sustainable constituents, which include recycled materials, naturally-derived materials and/or materials synthesized from bio-synthesized feedstock or bio-based materials. Moreover, these tires or tire components include threshold amounts of circular synthetic rubber while being characterized by high sustainable content. For example, the tires or tire components of the present invention can include greater than 40 wt %, in other embodiments greater than 50 wt %, and in other embodiments greater than 60 wt % sustainable materials. In these or other embodiments, the tire or tire components include from about 40 to about 90 wt %, in other embodiments from about 45 to about 85 wt %, and in other embodiments from about 50 to about 80 wt % sustainable material. In combination therewith, the rubber component of the tires or tire components of the present invention include greater than 10 wt %, in other embodiments greater than 20 wt %, in other embodiments greater than 30 wt %, in other embodiments greater than 40 wt %, in other embodiments greater than 45 wt %, and in other embodiments greater than 50 wt % circular synthetic rubber, which includes synthetic rubber produced according to embodiments of the present invention.

As indicated above, the vulcanizable compositions of this invention include a rubber component. This rubber component includes the circular synthetic rubber produced according to aspects of this invention. The rubber component may also include other synthetic rubber, such as synthetic rubber that derives from petroleum-based raw materials and has not been recycled, synthetic rubber that derives from other sustainable processes, as well as natural rubber. As the skilled person understands, natural rubber is synthesized by and obtained from plant life. For example, natural rubber can be obtained from Hevea rubber trees, guayule shrub, gopher plant, mariola, rabbitbrush, milkweeds, goldenrods, pale Indian plantain, rubber vine, Russian dandelions, mountain mint, American germander, and tall bellflower.

Other synthetic polymers, if used, can include, without limitation, synthetic polyisoprene, polybutadiene, polyisobutylene-co-isoprene, neoprene, poly(ethylene-co-propylene), poly(styrene-co-butadiene), poly(styrene-co-isoprene), poly(styrene-co-isoprene-co-butadiene), poly(isoprene-co-butadiene), poly(ethylene-co-propylene-co-diene), polysulfide rubber, acrylic rubber, urethane rubber, silicone rubber, epichlorohydrin rubber, and mixtures thereof. These elastomers can have a myriad of macromolecular structures including linear, branched, and star-shaped structures.

Generally, the rubber compositions of this invention include from about 30 to about 65 wt %, in other embodiments from about 35 to about 60 wt %, and in other embodiments from about 40 to about 55 wt % elastomer, based on the total weight of the tire component.

As suggested above, the rubber compositions include fillers such as organic and inorganic fillers. Examples of organic fillers include carbon black and starch. Examples of inorganic fillers include silica, aluminum hydroxide, magnesium hydroxide, mica, talc (hydrated magnesium silicate), and clays (hydrated aluminum silicates). In certain embodiments, a mixture of different fillers may be advantageously employed.

The amount of total filler employed in the rubber compositions can be up to about 150 parts by weight per 100 parts by weight of rubber (phr), with about 30 to about 125 phr, or about 40 to about 110 phr being typical. In certain embodiments the total filler content is greater than about 100 phr. In other embodiments, the total filler content is from about 50 to about 100 phr, and in in further embodiments from about 55 to about 95 phr.

Conventional carbon black can be used, which is generally known in the art. In one or more embodiments, carbon blacks include furnace blacks, channel blacks, and lamp blacks. More specific examples of carbon blacks include super abrasion furnace blacks, intermediate super abrasion furnace blacks, high abrasion furnace blacks, fast extrusion furnace blacks, fine furnace blacks, semi-reinforcing furnace blacks, medium processing channel blacks, hard processing channel blacks, conducting channel blacks, and acetylene blacks.

In particular embodiments, the carbon blacks may have a surface area (EMSA) of at least 20 m²/g and in other embodiments at least 35 m²/g; surface area values can be determined by ASTM D-1765 using the cetyltrimethylammonium bromide (CTAB) technique. The carbon blacks may be in a pelletized form or an unpelletized flocculent form. The preferred form of carbon black may depend upon the type of mixing equipment used to mix the rubber compound.

In one or more embodiments, carbon black can be sourced from a recycled material. Such recycled material can include reclaimed or recycled vulcanized rubber, whereby the vulcanized rubber is typically reclaimed from manufactured articles such as a pneumatic tire, an industrial conveyor belt, a power transmission belt, and a rubber hose. The recycled carbon black may be obtained by a pyrolysis process or other methods known for obtaining recycled carbon black. In an aspect, a recycled carbon black can be formed from incomplete combustion of recycled rubber feedstock or rubber articles. In another aspect, the recycled carbon black can be formed from the incomplete combustion of feedstock including oil resulting from the tire pyrolysis process. The carbon blacks utilized in the preparation of the vulcanizable elastomeric compositions can be in pelletized form or an unpelletized flocculent mass.

The amount of carbon black employed in the rubber compositions can be up to about 75 parts by weight per 100 parts by weight of rubber (phr), with about 5 to about 60 phr, or about 10 to about 55 phr being typical.

The rubber composition can further include filler in the form of one or more recycled rubbers in a particulate form. Recycled particulate rubber is typically broken down and reclaimed (or recycled) by any of a plurality of processes, which can include physical breakdown, grinding, chemical breakdown, devulcanization, cryogenic grinding, a combination thereof, etc. The term recycled particulate rubber can relate to both vulcanized and devulcanized rubber, where devulcanized recycle or recycled rubber (reclaim rubber) relates to rubber which has been vulcanized, ground into particulates and may have further undergone substantial or partial devulcanization. In an example, the recycled particulate rubber used in the rubber composition is essentially free of recycled rubber resulting from devulcanization. In a situation where the vulcanized rubber contains wire or textile fiber reinforcement, such wire or fiber reinforcement can be removed by any suitable process such as magnetic separation, air aspiration and/or air flotation step. In certain embodiments, the “recycled particulate rubber” comprises cured, i.e., vulcanized (crosslinked) rubber that has been ground or pulverized into particulate matter having a mean average particle size as discussed below.

Certain silicas may be considered sustainable materials. Some commercially available silicas which may be used as sustainable materials for the current invention include Hi-Sil™ 215, Hi-Sil™ 233, and Hi-Sil™ 190 (PPG Industries, Inc.; Pittsburgh, Pa.). Other suppliers of commercially available silica include Grace Davison (Baltimore, Md.), Degussa Corp. (Parsippany, N.J.), Rhodia Silica Systems (Cranbury, N.J.), and J.M. Huber Corp. (Edison, N.J.). Other sustainable silicas include those derived from rice husk ash.

In one or more embodiments, silicas may be characterized by their surface areas, which give a measure of their reinforcing character. The Brunauer, Emmet and Teller (“BET”) method (described in J. Am. Chem. Soc., 1939, vol. 60, 2 p. 309-319) is a recognized method for determining the surface area. The BET surface area of silica is generally less than 450 m²/g. Useful ranges of surface area include from about 32 to about 400 m²/g, about 100 to about 250 m²/g, and about 130 to about 240 m²/g, and about 170 to about 220 m²/g. In certain embodiments, the silica may have a BET surface area of 190 to about 280 m²/g. The pH's of the silicas are generally from about 5 to about 7 or slightly over 7, or in other embodiments from about 5.5 to about 6.8.

In one or more embodiments, where silica is employed as a filler (alone or in combination with other fillers), a coupling agent and/or a shielding agent may be added to the rubber compositions during mixing in order to enhance the interaction of silica with the elastomers. Useful coupling agents and shielding agents are disclosed in U.S. Pat. Nos. 3,842,111; 3,873,489; 3,978,103; 3,997,581; 4,002,594; 5,580,919; 5,583,245; 5,663,396; 5,674,932; 5,684,171; 5,684,172; 5,696,197; 6,608,145; 6,667,362; 6,579,949; 6,590,017; 6,525,118; 6,342,552; and 6,683,135; which are incorporated herein by reference.

The amount of silica employed in the rubber compositions can be from about 1 to about 150 phr or in other embodiments from about 5 to about 130 phr. The useful upper range is limited by the high viscosity imparted by silicas. In certain embodiments, the silica employed in the rubber composition is derived from rice husk ash only, and in other embodiments the rubber compositions do not include silica from non-rice husk ash derived processes. When silica is used together with carbon black, the amount of the silica or carbon black individually can be as low as about 1 phr. Generally, the amounts of coupling agents and shielding agents range from about 4 wt % to about 20 wt % based on the weight of silica used. In one or more embodiments, where carbon black and silica are employed in combination as a filler, the weight ratio or silica to total filler may be from about 5 wt % to about 99 wt % of the total filler, in other embodiments from about 10 wt % to about 90 wt % of the total filler, or in yet other embodiments from about 50 wt % to about 85 wt % of the total filler. In certain embodiments the silica and carbon black fillers employed in the rubber composition are selected from the group consisting of sustainable pyrolysis carbon black and/or rice husk ash derived silica.

A multitude of rubber curing agents (also called vulcanizing agents) may be employed, including sulfur or peroxide-based curing systems. Curing agents are described in Kirk-Othmer, ENCYCLOPEDIA OF CHEMICAL TECHNOLOGY, Vol. 20, pgs. 365-468, (3^{rd} Ed. 1982), particularly Vulcanization Agents and Auxiliary Materials, pgs. 390-402, and A.Y. Coran, Vulcanization, ENCYCLOPEDIA OF POLYMER SCIENCE AND ENGINEERING, (2^{nd} Ed. 1989), which are incorporated herein by reference. Vulcanizing agents may be used alone or in combination.

Other ingredients that are typically employed in rubber compounding may also be added to the rubber compositions. These include accelerators, accelerator activators, oils, plasticizer, waxes, scorch inhibiting agents, processing aids, zinc oxide, tackifying resins, reinforcing resins, fatty acids such as stearic acid, peptizers, and antidegradants such as antioxidants and antiozonants.

With regard to oils, sustainable oils, which include plant-based oils and bio-based oils, may be used. Plant-based oils may include plant-based triglycerides. Exemplary oils include, without limitation, palm oil, soybean oil (also referred to herein as soy oil), rapeseed oil, sunflower seed, peanut oil, cottonseed oil, oil produced from palm kernel, coconut oil, olive oil, corn oil, grape seed oil, hemp oil, linseed oil, rice oil, safflower oil, sesame oil, mustard oil, flax oil. Other examples include nut-derived oils such oils obtained from beech nuts, cashews, mongongo nuts, macadamia nuts, pine nuts, hazelnuts, chestnuts, acorns, almonds, pecans, pistachios, walnuts, or brazil nuts. As the skilled person will appreciate, these oils can be produced by any suitable process such as mechanical extraction (e.g., using an oil mill), chemical extraction (e.g., using a solvent, such as hexane or carbon dioxide), pressure extraction, distillation, leaching, maceration, purification, refining, hydrogenation, sparging, etc.

Bio-based oils, also referred to as bio-oils, can include oils produced by a recombinant cell. For example, bio-oils produced by recombinant cells can be produced using a select strain of algal cells that are fed with a supply of sugars (e.g., sucrose) and then allowed to ferment and produce a bio-oil with a selected profile; after sufficient growth or fermentation has taken place, the bio-oil is isolated from the cells and collected.

Generally, the rubber compositions of this invention can include from about 1 to about 70 parts by weight, or in other embodiments from about 5 to about 50 parts weight total oil per 100 parts by weight rubber. The amount of sustainable oil, relative to the total weight of oil included, may be from about 1 wt % to about 99 wt %, or in other embodiment from about 20 wt % to about 80 wt %.

With regard to waxes, the rubber compositions can include one or more sustainable waxes, which include natural waxes. A natural wax, or one with no petroleum as its raw material, can include carnauba wax, candelilla wax (e.g., extracted from candelilla flowers), rice wax (e.g., separated from rice bran oil) and Japan wax (e.g., extracted from Japanese wax tree).

Generally, the rubber compositions of this invention include from about 1 to about 20 parts by weight, or in other embodiments from about 2 to about 15 parts by weight total wax per 100 parts by weight rubber. The amount of sustainable wax, relative to the total weight of wax included, may be from about 1 wt % to about 99 wt %, or in other embodiment from about 20 wt % to about 80 wt % of the total wax. In certain embodiments, the rubber composition includes sustainable waxes only.

All ingredients of the rubber compositions can be mixed with standard mixing equipment such as Banbury or Brabender mixers, extruders, kneaders, and two-rolled mills. In one or more embodiments, the ingredients are mixed in two or more stages. In the first stage (often referred to as the masterbatch mixing stage), a so-called masterbatch, which typically includes the rubber component and filler, is prepared. To prevent premature vulcanization (also known as scorch), the masterbatch may exclude vulcanizing agents. The masterbatch may be mixed at a starting temperature of from about 25° C. to about 125° C. with a discharge temperature of about 135° C. to about 180° C. Once the masterbatch is prepared, the vulcanizing agents may be introduced and mixed into the masterbatch in a final mixing stage, which is typically conducted at relatively low temperatures so as to reduce the chances of premature vulcanization. Optionally, additional mixing stages, sometimes called remills, can be employed between the masterbatch mixing stage and the final mixing stage. One or more remill stages are often employed where the rubber composition includes silica as the filler. Various ingredients including the polymers of this invention can be added during these remills.

The mixing procedures and conditions particularly applicable to silica-filled tire formulations are described in U.S. Pat. Nos. 5,227,425; 5,719,207; and 5,717,022, as well as European Patent No. 890,606, all of which are incorporated herein by reference. In one embodiment, the initial masterbatch is prepared by including the polymer and silica in the substantial absence of coupling agents and shielding agents.

In order to fabricate tire components with the polymers produced by the invention, the skilled person appreciates that the polymers are mixed with various other ingredients (e.g. filler and curative) to produce a rubber composition (also referred to as vulcanizable composition), and the vulcanizable composition is then processed into tire components according to ordinary tire manufacturing techniques, which generally include standard rubber shaping and molding techniques. The tire components may include, but are not limited to, tire treads, sidewalls, subtreads, body ply skims, and bead filler. The various tire components are then assembled into a green tire (i.e. an uncured tired), placed within a mold, and then vulcanized. Typically, vulcanization is effected by heating the vulcanizable composition in a mold; e.g., it may be heated to about 140° C. to about 180° C. Cured or crosslinked rubber compositions may be referred to as vulcanizates, which generally contain three-dimensional polymeric networks that are thermoset. The other ingredients, such as fillers and processing aids, may be evenly dispersed throughout the crosslinked network. Pneumatic tires can be made as discussed in U.S. Pat. Nos. 5,866,171; 5,876,527; 5,931,211; and 5,971,046, which are incorporated herein by reference.

In one or more embodiments, the tires can include fabric reinforcement made by using non-petroleum materials in place of synthetic fibers. For example, mechanical recycled fibers, chemical recycled fibers, or bio-based fibers can be used. Likewise, the tires can include metal reinforcement made from recycled steel and/or other circular or sustainable metals. These non-petroleum fabrics and recycled metals can be used exclusively within the tires or in combination with traditional fabric and/or metal reinforcement.

Various modifications and alterations that do not depart from the scope and spirit of this invention will become apparent to those skilled in the art. This invention is not to be duly limited to the illustrative embodiments set forth herein.

1. A process comprising:
(a) providing a feedstock that includes carbonaceous materials;
(b) gasifying the feedstock to produce a gaseous stream including carbon monoxide, hydrogen, and carbon dioxide;
(c) converting at least a portion of the carbon monoxide, hydrogen, and carbon dioxide to methanol;
(d) converting at least a portion of the methanol to butene; and
(e) converting at least a portion of the butene to butadiene monomer.

2. The process of any of the preceding claims claim 1, where the step of converting includes preferentially producing the methanol relative to other potential products.

3. The process of claim 1, where the step of converting at least a portion of the carbon monoxide, hydrogen, and carbon dioxide to methanol includes supplementing the carbon monoxide, hydrogen, and carbon dioxide with an amount of hydrogen tailored to the production of the methanol.

4. The process of claim 1, where the step of converting at least a portion of the carbon monoxide, hydrogen, and carbon dioxide to methanol includes supplementing the carbon monoxide, hydrogen, and carbon dioxide with an additive tailored to the production of the methanol.

5. The process of claim 1, where the step of converting at least a portion of the methanol to butene preferentially produces the butene relative to other olefins.

6. The process of claim 1, where the step of converting at least a portion of the methanol to butene includes operation at a particular temperature tailored to the production of the butene relative to other olefins.

7. The process of claim 1, where the feedstock includes used tire feedstock.

8. The process of claim 1, where the feedstock includes biomass.

9. The process of claim 1, where the feedstock includes municipal solid waste.

10. The process of claim 1, where the feedstock includes a mixture of tire feedstock and a complementary feedstock.

11. The process of claim 1, further comprising the step of polymerizing the butadiene monomer to thereby produce a polymer that is a polybutadiene polymer.

12. The process of claim 11, further comprising the step of providing comonomer, and where said step of polymerizing the butadiene monomer includes copolymerizing the butadiene monomer with the comonomer to thereby produce a polymer that is a polybutadiene copolymer.

13. The process of claim 12, where the comonomer is a sustainable comonomer.

14. The process of claim 11, further comprising the step of fabricating a tire component with the polymer.

15. A method for producing an elastomeric polymer, the method comprising:
(a) providing 1,3-butadiene monomer synthesized from constituents of a gaseous stream that is obtained from the gasification of a feedstock including carbonaceous materials; and
(b) polymerizing the 1,3-butadiene monomer to form a polymer.

16. The method of claim 15, where the gaseous stream includes carbon monoxide, hydrogen, and carbon dioxide, and wherein the 1,3-butadiene monomer is formed by converting at least a portion of the carbon monoxide, hydrogen, and carbon dioxide to methanol, converting at least a portion of the methanol to butene; and converting at least a portion of the butene to butadiene monomer.

17. (canceled)

18. (canceled)

19. (canceled)

20. The method of claim 15, where the carbonaceous material includes used tire feedstock.

21. The method of claim 15, where the carbonaceous material includes biomass.

22. The method of claim 15, where the carbonaceous material includes solid municipal waste.

23. (canceled)

24. A tire component comprising:
a cured rubber matrix with filler dispersed therein, where the cured rubber matrix includes greater than 40 wt % of a crosslinked circular synthetic rubber that is obtained by polymerizing monomer synthesized from constituents of a gaseous stream that is obtained from the gasification of a feedstock including carbonaceous materials.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
